(19) United States
(12) Patent Application Publication
(10) Pub. No.: US 2021/0286864 A1
(43) Pub. Date: Sep. 16, 2021

(54) DUAL-MODE BIOMETRIC CONFIGURATION FOR USER VALIDATION AND USER HEALTH CHECK TO DETERMINE ACCESS TO PRODUCTS AND/OR SERVICES

(71) Applicant: NextGen Monetization Trust, Newark, DE (US)

(72) Inventor: Christopher John Burke, Central (HK)

(73) Assignee: NextGen Monetization Trust, Newark, DE (US)

(21) Appl. No.: 16/817,844

(22) Filed: Mar. 13, 2020

(51) Int. Cl. G06F 21/32; G16H 40/60

(52) U.S. Cl. CPC G06F 21/32; G16H 40/60

(57) ABSTRACT

A process receives, at a mobile computing device, a biometric identification input from a user. Furthermore, the process compares, with a processor at the mobile computing device, the biometric identification input with a previously-stored biometric identification input to validate an identity of the user. Moreover, the process measures, at the mobile computing device, one or more health parameters of the user. Finally, the process sends, based upon the validation of the identity of the user, the one or more health measurements to an access device that grants access to the user to a product or service based upon the one or more health measurements complying with one or more health criteria to validate a health check of the user.

### BACKGROUND

### 1. Field

This disclosure generally relates to the field of biometric devices. More particularly, the disclosure relates to biometric analysis of a user.

### 2. General Background

Spanning millennia to the present, communities of varying sizes (workplaces, colleges, universities, villages, towns, cities, states, provinces, nations, etc.) have faced recurring social policy challenges. In any community-based environment, the core concern is typically managing the health and well-being of the group of human beings in that environment. Although many community-based protocols have provided numerous benefits in the way of basic and essential services (food, water, shelter, electricity, plumbing, transportation, etc.), they have been lacking in other areas.

A particular challenge in a community-based environment is virus transmission: human beings are susceptible to various forms of illness based on living and working with other human beings, who may have been infected with a virus. A common response by entities (e.g., governments, hospitals, etc.) managing community protocols is reactionary, namely manufacturing and prescribing medications to address symptoms after people have actually obtained the virus. As an example, millions of people in the United States get the common cold each year, and have to take numerous days off of work, leading to reductions in work productivity. Even though the common cold spreads so quickly from person-to-person, affecting so many people year-after-year, the vast majority of over-the-counter medications are directed toward symptom relief/mitigation, rather than preventing virus transmission. And given that prescribed medications, such as antibiotics, are typically ineffective against viruses, such as the rhinovirus that is one of the many viruses that can lead to the common cold, the reactionary approach has not mitigated the transmission of the common cold from human-to-human.

To further exacerbate matters, a number of viruses typically lead to health complications that extend beyond losses in work productivity. In particular, viruses such as influenza and corona virus disease nineteen (“COVID-19”) have threatened the lives of many people on a global scale. In the case of influenza, vaccines are typically manufactured on a yearly basis, but their efficacy is often lackluster (e.g., often less than fifty percent; sometimes even less than thirty percent); a contributing factor being mutations of the virus that essentially work around the vaccine. And in the case of COVID-19, no vaccine is currently available.

Moreover, community-based management has significant other challenges besides virus transmission. As an example, addiction to prescribed opioid medications has reached a crisis level in the United States; in many instances leading to overdose. The problem oftentimes may not be a result of the initial issuance of the prescription, but rather the subsequent usage of the prescription by the patient. For instance, a patient using more than the prescribed dosage, or taking the medication in a manner that was unintended by the prescribing physician (e.g., crushing a pill for snorting), may lead to an overdose. As a few other examples, firearm usage, drunk driving, and reckless driving have all led to many fatalities, instilling fear in communities on a national scale.

Accordingly, community-based environments currently do not have effective systems in place for effectively managing community protocols in an optimal manner to minimize risks, from a variety of sources, to the health and well-being of community members.

### SUMMARY

In one aspect of the disclosure, a process receives, at a mobile computing device, a biometric identification input from a user. Furthermore, the process compares, with a processor at the mobile computing device, the biometric identification input with a previously-stored biometric identification input to validate an identity of the user. Moreover, the process measures, at the mobile computing device, one or more health parameters of the user. Finally, the process sends, based upon the validation of the identity of the user, the one or more health measurements to an access device that grants access to the user to a product or service based upon the one or more health measurements complying with one or more health criteria to validate a health check of the user.

In another aspect of the disclosure, the process sends, with a processor at the mobile computing device, the biometric identification input to an access device to obtain identification validation of the user, in addition to health criteria validation.

In yet another aspect of the disclosure, a computer program product is provided. The computer program product comprises a non-transitory computer useable storage device having a computer readable program, which when executed on the computing device causes the computing device to perform the foregoing process. Alternatively, an apparatus may implement the foregoing processes.

### BRIEF DESCRIPTION OF THE DRAWINGS

The above-mentioned features of the present disclosure will become more apparent with reference to the following description taken in conjunction with the accompanying drawings wherein like reference numerals denote like elements and in which:

FIG. 1A illustrates a cloud-based configuration that utilizes an application server to perform processing of one or more sets of biometric data to validate a user's identity and establish a preliminary health check for that user.

FIG. 1B illustrates that the user access database may be integrated within the mobile computing device.

FIG. 1C illustrates the product/service provider access device transmitting the preliminary health check data to a product/service provider server, which may be remotely situated from the product/service provider, that assesses the compliance of the preliminary health check data.

FIG. 1D illustrates the mobile computing device transmitting both layers of biometric data to the product/service provider access device for transmission to the product/service provider server, which may perform both user validation and preliminary health check compliance.

FIG. 2A illustrates an example of the user using the mobile computing device in conjunction with a smart bracelet.

, FIG. 2B illustrates a smart wearable device in the form of a smart hat that measures and emits the preliminary health check data.

FIG. 2C illustrates a smart wearable device in the form of smart shoes, which may have a built-in thermometer that measures the foot temperature of the user.

FIG. 3 illustrates a statically-positioned health measurement device that may measure the preliminary health check data of the user.

FIG. 4 illustrates a system configuration for the computing device, illustrated in FIGS. 1A-1D.

FIG. 5A illustrates a bus, which initially has restricted access, as indicated by an access indicium.

FIG. 5B illustrates the user holding the mobile computing device to his or her ear to perform a preliminary health check.

FIG. 5C illustrates the door of the bus automatically opening to grant access to the user upon compliance with the health criteria.

FIG. 6A illustrates an airline representative asking to view an e-ticket, as well as to perform biometric validation of the identity of the user.

FIG. 6B illustrates the user 102 being cleared to board the airplane upon compliance with both the identification and health validation.

FIG. 7A illustrates the user validating his identity, via a biometric input at the mobile computing device.

FIG. 7B illustrates the user using a statically-positioned blood pressure monitoring device to measure his blood pressure.

FIG. 7C illustrates a pharmacist providing the medication to the user upon the biometric validation criteria being met.

FIG. 8A illustrates the user entering the firearm distribution environment to purchase a firearm.

FIG. 8B illustrates a point of sale (“POS”) device indicating that the identification and health validations were successful, thereby allowing the firearm representative to proceed with the sale of the firearm to the user.

FIG. 9A illustrates the user approaching a turnstile to obtain access to a concert.

FIG. 9B illustrates the user successfully completing the biometric identification and biometric health check for automatic, permitted access through the turnstile.

FIG. 10A illustrates the user utilizing a graphical user interface (“GUI”) on the mobile computing device to access a menu to unlock the automobile.

FIG. 10B illustrates the mobile computing device being configured to render a visual game, which tests the reflexes of the user, as a biometric health check.

FIG. 10C illustrates the user attempting to open a door of the automobile, after successful biometric identification and health check validation.

FIG. 10D illustrates the user successfully opening the door of the automobile.

FIG. 11A illustrates the user in the automobile driving toward a freeway access point.

FIG. 11B illustrates a biometric health check of the user being performed to ensure that the user is in a condition to drive the automobile.

FIG. 12A illustrates the user utilizing the mobile computing device to emit location data, biometric identification data, and biometric health data to an unmanned aerial vehicle (“UAV”).

FIG. 12B illustrates the UAV delivering the package upon successful biometric identification and biometric health check validation.

FIG. 13A illustrates the user approaching a vending machine to provide biometric identification.

FIG. 13B illustrates the user obtaining his or her product from the vending machine upon compliance with the one or more health criteria.

FIG. 14 illustrates a process that may be utilized to implement a dual-biometric configuration.

### DETAILED DESCRIPTION

A dual-mode biometric configuration is provided to accomplish at least two objectives prior to providing access to products and/or services: user identification validation and health check validation. In contrast with the reactionary approach of previous community-based protocols, the dual-mode biometric configuration is preemptive in identifying symptoms that should lead to a denial of access to products and/or services for the overall well-being of a community. Although virus strains may vary significantly in quantity, potency, and mutations, the number of symptoms indicative of a potential threat to a community is relatively small by comparison. For example, a person with a temperature of over one hundred four degrees is in no condition to be going to work, using public transportation, or driving a vehicle, irrespective of the underlying condition that caused such symptoms in that person. Given the exponential rate of viral infection of many viruses (e.g., common cold, influenza, COVID-19, etc.), preventing exposure of one contagious person to even a small group of people may prevent thousands of infections.

In one embodiment, a software application is specifically configured to operate on a user's mobile computing device (e.g., smartphone, tablet device, smartwatch, smart bracelet, smart necklace, smart apparel, etc.) for performing biometric validation of a user with respect to identification and health check. The mobile computing device, itself, may perform the biometric identification validation by comparing the biometric data inputted by the user with biometric data of the user previously stored by the mobile computing device. As a result of such localized biometric validation, the software application may allow for access and external transmission of data associated with the validated user (e.g., e-tickets, access codes, medication prescriptions, payment data, etc.). In addition, the software application may conduct, via one or more integrated or external health monitoring devices, a preliminary health check on the user simultaneously, or in real-time (i.e., as measured by a humanly imperceptible delay) with, the biometric identification validation of the user. The software application may then be configured to transmit user access data with the preliminary health check data to a provider of a product or service, which may then determine, based on its own healthcare management protocols, whether or not to allow the product or service to be provided to the user. In another embodiment, the software application is configured to internally determine compliance with the healthcare management protocol. For instance, one set of health check parameters for one person may have significantly different meaning for that of another. The software application may customize the preliminary health check according to the specific user of the mobile computing device.

In another embodiment, the biometric validation and preliminary health check may be performed external to the mobile computing device. For example, a product provider may be required by regulation to confirm the identity of the user. Accordingly, the product provider system may receive the biometric data from the mobile computing device of the user to perform a comparison with a remote database of biometric data.

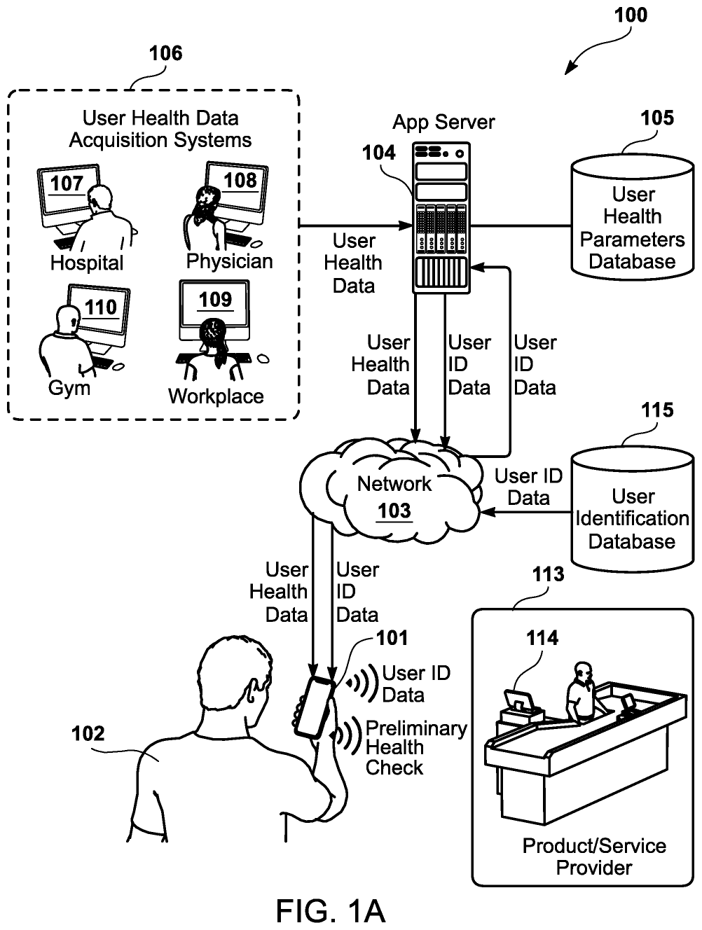

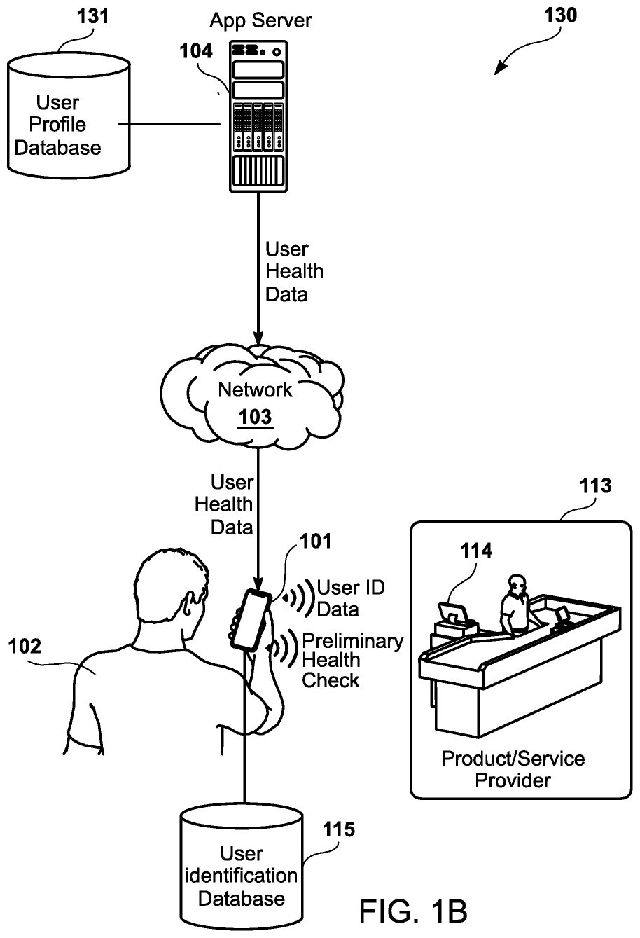

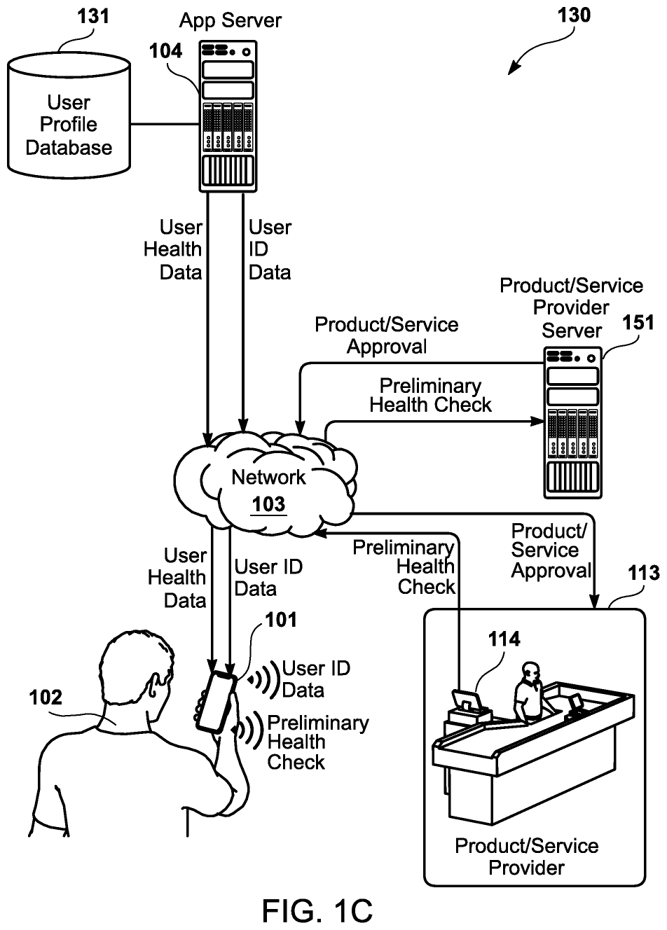

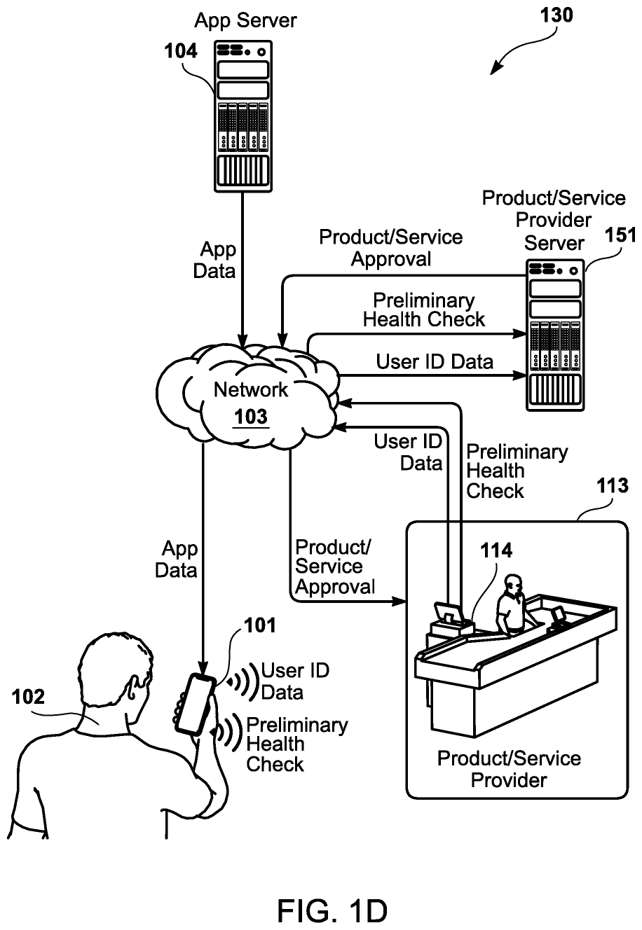

FIGS. 1A-1D illustrate various dual-mode biometric configurations of user mobile computing device 101. In particular, FIG. 1A illustrates a cloud-based configuration 100 that utilizes an application server 104 to perform processing of one or more sets of biometric data to validate a user's identity and establish a preliminary health check for that user. Without the limiting the applicability of the cloud-based configuration 100 to various contexts, the cloud-based configuration 100, in its essence, uses biometric data to allow for a user 102, via a mobile computing device 101, to obtain access to a product or service from a product/service provider 113. (The product/service provider 113 is illustrated as being operated by a user, but may be partially, or completely, operated autonomously. For instance, the product/service provider 113 may be an automated facility, kiosk, vending machine, automobile, UAV, etc.) Furthermore, in order to obtain the product or service for the user 102, the user mobile computing device 101 may provide two layers of biometric data, via proximity-based wireless communication (e.g., Near Field Communication (“NFC”), radio frequency identification (“RFID”), BLUETOOTH, etc.), to a product/service provider access device 114.

With respect to the cloud-based configuration 100, the application server 104 may be in operable communication with various health data acquisition systems 106 (e.g., hospital system 107, physician system 108, workplace system 109, gym system 110, etc.), which are instructed to obtain various health parameters with respect to the user 102. (Alternatively, or in addition, the application server 104 may also receive health parameters pertaining to the user 102 from a wearable device directly worn by the user 102.) In essence, the health parameters obtained from the user health data acquisition systems 106 may establish a baseline of health data for the particular user 102. Furthermore, the health parameters may include additional information, such as electronic medical records, medication prescriptions, physician instructions, etc. In one embodiment, the health parameters of the user 102 may be stored in a user health parameters database 105 in a centralized location for fast search and access by the application server 104.

In addition, in one embodiment, the application server 104 may be in operable communication with a user identification database 115, which stores the biometric data necessary to validate the particular user 102 at a given product/service provider 113 prior to the product/service provider 113 granting access to the product or service to the user 102. For example, the user access database 115 may store a fingerprint of the user 102, and send that fingerprint data to the mobile computing device 101 at the point of access so that the mobile computing device 101 may compare that fingerprint with a fingerprint received at the mobile computing device 101. In essence, the mobile computing device 101 may validate that the user 102 is who he or she says he or she is prior to the product/service provider 113 granting access to the product or service. (The phrase “granting access” is intended to encompass a sale or a removal of a restriction on an item or service, whether currently owned by the user 102 or not.)

In an alternative, embodiment, the user identification database 115 is integrated within the mobile computing device 101, as illustrated in FIG. 1B. In other words, the mobile computing device 101 may locally store all of the user identification data of the user 102, without any external exposure of that access data to remotely situated systems. As a result, the user 102 may maintain the privacy of his or her data, while still allowing for his or her identify to be confirmed. In other words, the mobile computing device 101 stores the biometric identification data (iris scan, fingerprint, thumbprint, palm scan, etc.) of the user 102. Upon receiving a user input of biometric data at the point of access for the product/service, the mobile computing device 101 may perform a comparison of the biometric data to validate the identity of the user 102. Just as illustrated in FIG. 1A, the mobile computing device in FIG. 1B may transmit a proximity-based signal to the product/service provider access device 114 indicating that biometric identification has been validated. As an example, the mobile computing device 101 may transmit an image of a photo id along with a biometric identification approval signal to the product/service provider access device 114. In another embodiment, the biometric validation performed in FIGS. 1A and 1B is only used by the mobile computing device 101 to release preliminary health check biometric data to the product/service provider access device 114, not for identification.

Additionally, the application server 104 may be in operable communication with a user profile database 131. For example, the user 102, himself or herself, may provide the user health parameters as inputs via the mobile computing device 101, for storage in the user profile database 131. In one embodiment, the user 102 provides users inputs (e.g., virtual keyboard inputs, swipes, gestures, etc.) to input the health parameters. In another embodiment, the user 102 invokes the mobile computing device 101 to capture the health parameters for the user 102. For example, the user 102 may use an image capture device integrated within the mobile computing device 101 to capture baseline user data, such as pupil dilation, body temperature, etc.

In one embodiment, as illustrated in FIGS. 1A and 1B, the product/service provider 113 may receive the preliminary health check data from the mobile computing device 101, and determine, either automatically via the product/service provider access device 114 or manually by a product/service provider representative, whether the preliminary health check complies with one or more criteria for product/service access. For example, the product/service provider 113 may have established its own preliminary health check thresholds (e.g., body temperature, blood pressure, pulse rate, pupil dilation, sweat detection, etc.), or follow those of a regulatory entity, which have to be met prior to granting access of a product/service to the user 102. In another embodiment, as illustrated in FIG. 1C, the product/service provider access device 114 transmits the preliminary health check data to a product/service provider server 151, which may be remotely situated from the product/service provider 113, that assesses the compliance of the preliminary health check data. The product/service provider server 151 may then provide an instruction to the product/service provider access device 114 as to whether the product/service provider access device 114 should grant access to the product/service to the user 102.

Finally, as illustrated in FIG. 1D, the mobile computing device 101 may transmit both layers of biometric data to the product/service provider access device 114 for transmission to the product/service provider server 151, which may perform both user validation and preliminary health check compliance; upon a successful determination for both, the product/service provider server 151 may provide an instruction to the product/service provider access device 114 to grant the user 102 access to the product or service.

With any of the configurations provided for in FIGS. 1A-1D, the mobile computing device 101 may communicate, via a network 103, with the application server 104 to obtain data for rendering of a software application. The user 102 may interact with the software application to perform tasks, such as establishing health baseline data, biometric identification validation, and user inputs for preliminary health checks.

In essence, the mobile computing device 101 illustrated in FIGS. 1A-1D is an integrated mobile device that has componentry encompassed therein to process both layers of biometric data: identification data and preliminary health check data. For example, the mobile computing device 101 may have a touch-based display screen, which can receive thumbprint data to identify the user 102, and an infrared (“IR”) sensor that may detect the temperature of the user 102. As a result, the user 102 may use just one device to obtain access to various products and/or services. In one embodiment, each set of biometric data is associated with a distinct user input (i.e., a thumbprint for identification and an ear scan for temperature). In another embodiment, one user input may encompass both sets of biometric data (e.g., a facial recognition scan may be performed to obtain facial data points as well as measure the forehead temperature of the user 102).

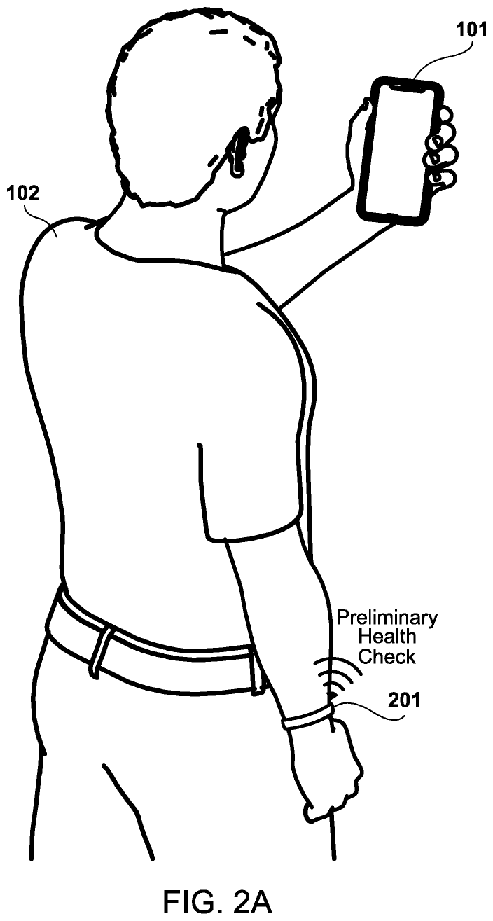

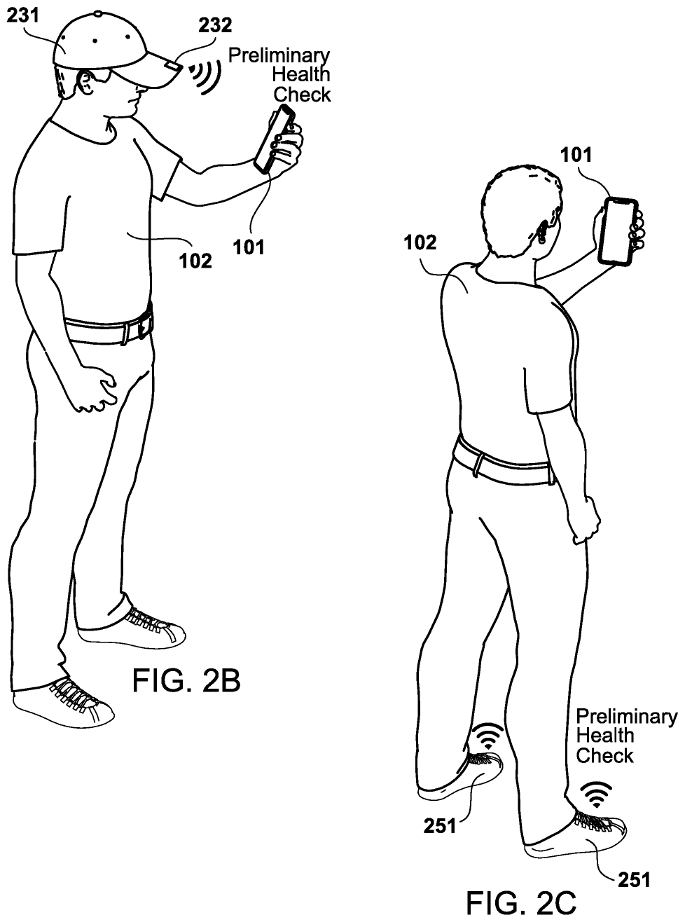

By way of contrast, the mobile computing device 101 illustrated in FIGS. 2A-2C may wirelessly (e.g., via a BLUETOOTH link of biometric card interface stored within the mobile computing device 101) communicate with one or more accessory devices carried or worn by the user 102. In particular, FIG. 2A illustrates an example of the user 102 using the mobile computing device 101 in conjunction with a smart bracelet 201. In essence, the user 102 may provide the user identification data via one device (e.g., mobile computing device 101) and provide the preliminary health check data via another device (e.g., smart bracelet 201). Furthermore, the smart bracelet 201 may be configured to automatically emit the preliminary health check data to the product/service provider access device 114, illustrated in FIGS. 1A-1D, upon user validation via the biometric identification data received at the mobile computing device 101. Accordingly, the user 102 may not have to press any buttons on, or perform any other actions with respect to, the smart bracelet 201. In another embodiment, the smart bracelet 201 is configured to automatically emit the preliminary health check data based upon location data sensed by the smart bracelet 201 or the mobile computing device 101. For example, the smart bracelet 201 may be configured to emit a particular form of preliminary health check data (e.g., blood pressure) based upon detection, such as a via a global position system (“GPS”) sensor, of the user 102 being geolocated at a pharmacy. (The GPS may be integrated in either, or both of, the smart bracelet 201 or the mobile computing device 101.)

The smart bracelet 201 is just an example of a smart wearable device that may be worn by the user 102 to determine the preliminary health check data. As another example, FIG. 2B illustrates a smart wearable device in the form of a smart hat 231 that measures and emits the preliminary health check data. For instance, the smart hat 231 may have a built-in thermometer that measures the forehead temperature of the user 102. A transmitter 232 situated on the brim of the cap, or integrated within the smart hat 231 in a manner that does not draw attention, may send the preliminary health check data to the mobile computing device 101. As yet another example, FIG. 2C illustrates a smart wearable device in the form of smart shoes 251, which may have a built-in thermometer that measures the foot temperature of the user 102.

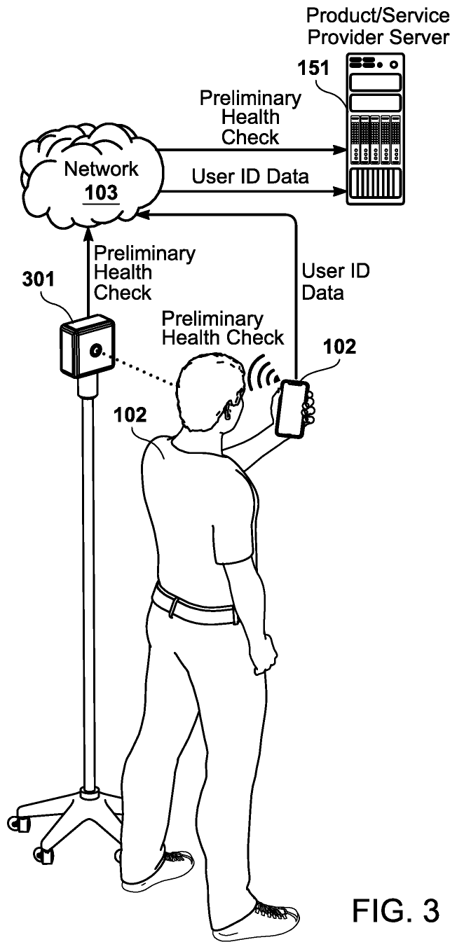

Furthermore, the preliminary health check data is not limited to being generated at the mobile computing device 101 or a smart wearable device worn by the user 102. For example, as illustrated in FIG. 3, a statically-positioned health measurement device 301 may be measure the preliminary health check data of the user 102. For instance, the statically-positioned health measurement device 301 may scan an eardrum or forehead of the user to determine if the user 102 has a fever. In one embodiment, the statically-positioned health measurement device 301 transmits the preliminary health check data, via the network 103, to the product/service provider server 151. Furthermore, the mobile computing device 101 may also transmit the user identification data, via the network 103, to the product/service provider server 151. In another embodiment, the statically-positioned health measurement device 301 may transmit the preliminary health check data to the mobile computing device 101, which may then send both, or either of, the biometric identification data and the biometric health check data to the product/service provider server 151. In yet another embodiment, the mobile computing device 101 may transmit the biometric identification data to the statically-positioned health measurement device 301, which may then send both, or either of, the biometric identification data and the biometric health check data to the product/service provider server 151.

The statically-positioned health measurement device 301 may be positioned unobtrusively so that it is not apparent to the user 102. For example, it may be integrated into a wall, turnstile, or other statically-positioned structure.

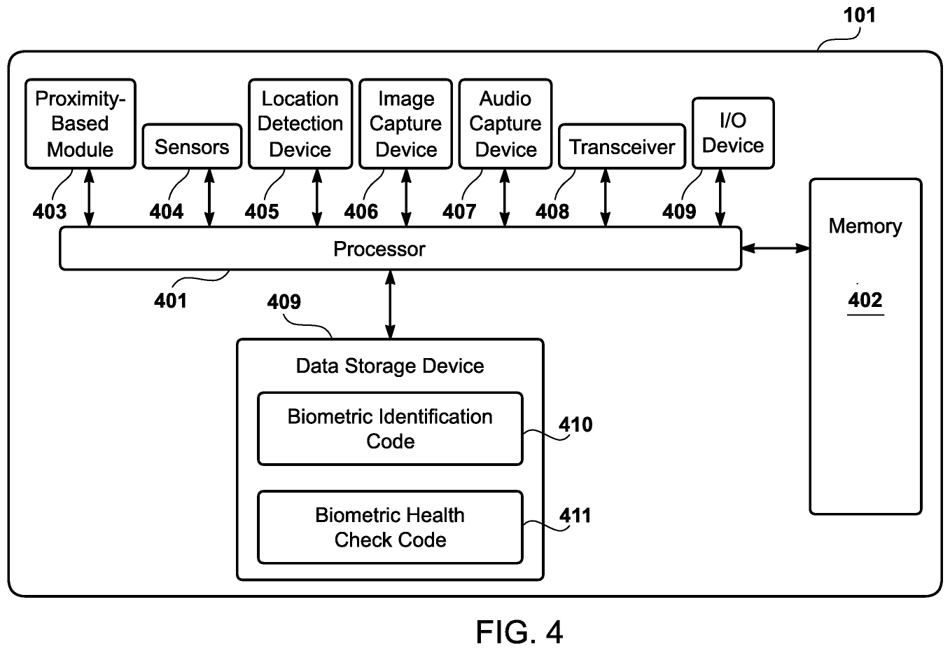

Moreover, FIG. 4 illustrates a system configuration for the computing device 101, illustrated in FIGS. 1A-1D. A processor 401 may be specialized for biometric operations and GUI generation.

The system configuration may also include a memory device 402, which may temporarily store data structures used by the processor 401. As examples of such data structures, a data storage device 409 may store biometric identification code 410 and biometric health check code 411. The processor 401 may execute the biometric identification code 410 and biometric health check code 411 to perform dual layers of biometric validation (i.e., identification and health check) to obtain access to a product or service for the user 102. Alternatively, one, or both, of the biometric identification code 410 and biometric health check code 411 may be executed by another device/system (e.g., server, wearable accessory device, etc.) in operable communication with the mobile computing device 101.

In one embodiment, the processor 401 is in operable communication with a proximity-based module 403, which is a physical circuit, such as an NFC physical circuit. Upon detecting the presence of an NFC-based reader within the product/service access device 114, the NFC-based module 403 awaits an indication of biometric validation from the processor 401, at which time the NFC-based circuit transitions from an open position to a closed position to transmit data (identification, health check, or both), via magnetic inductive communication, to the NFC-based reader within the product/service access device 114. In another embodiment, the proximity-based module 403 is a logical circuit that is implemented via software. Furthermore, the proximity-based module 403 may perform its functionality via two sub-modules, a proximity-based detection module and a proximity-based transmission module, or as one unified module. (The example of NFC is only one example, and is not intended to limit the applicability of the configurations provided for herein to the exclusion of other proximity-based technologies.)

Moreover, the mobile computing device 101 may have one or more sensors 404, image capture devices 406, and audio capture devices 407, specifically configured to sense health parameters pertaining to the user 102. For example, the sensors 404 may be IR sensors that sense body temperature. As another example, the image capture devices 406 may capture imagery of pupil dilation. As yet another example, the audio capture device 407 may capture audio of breathing patterns of the user 102. The processor 401 may then perform audio analysis to determine health symptoms, such as congestion, coughing, wheezing, etc.

Additionally, the mobile computing device 101 may have a location-based detection device 405, such as a GPS device. Based on the detection of various locations, the processor 401 may perform a lookup in a configuration table, stored in the memory 402, to determine an associated biometric identification modality (e.g., iris scan, fingerprint, thumb scan, palm scan, facial recognition, etc.) for that geographic location. For example, a pharmacy may necessitate biometric validation via an iris scan, whereas a firearm shop may necessitate biometric validation via a thumbprint. The configuration table may provide for an automatic determination by the processor 401 of the biometric modality that should correspond to the location detected by the location-based detection device 405.

Finally, the mobile computing device 101 may have one or more input/output (“I/O”) devices 409, which may receive inputs and provide outputs, and a transceiver 408 to send and receive data. (Alternatively, a separate transmitter and receiver may be used instead.) Various devices (e.g., keyboard, microphone, mouse, pointing device, hand controller, joystick, etc.) may be used for the I/O devices 409.

Although the system configuration is described with respect to the mobile computing device 101, alternatively, it may be utilized in whole, or in part, by a server, such as the application server 104 or the product/service provider server 151 illustrated in FIGS. 1C and 1D, or a smart accessory to the mobile computing device 101, such as the smart bracelet 201, smart hat 231, or smart shoes 251 illustrated in FIGS. 2A-2C.

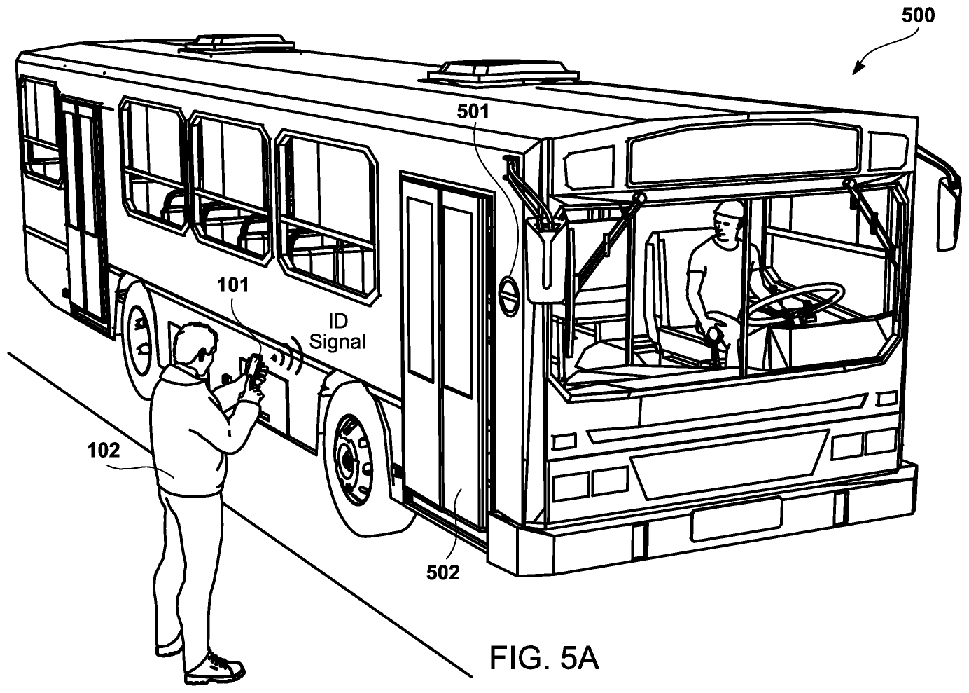

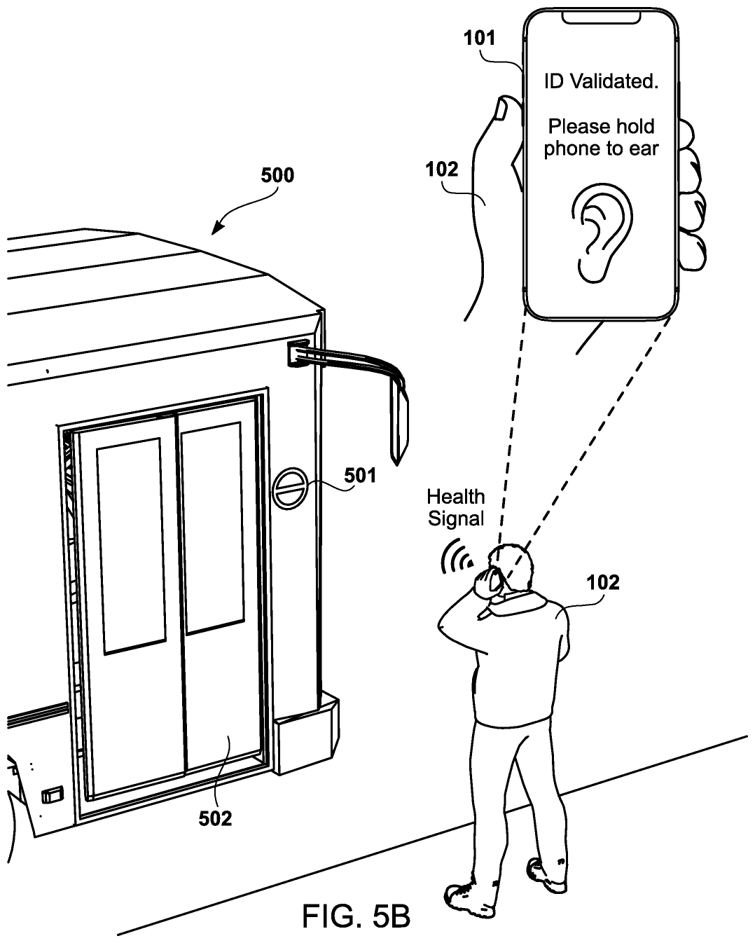

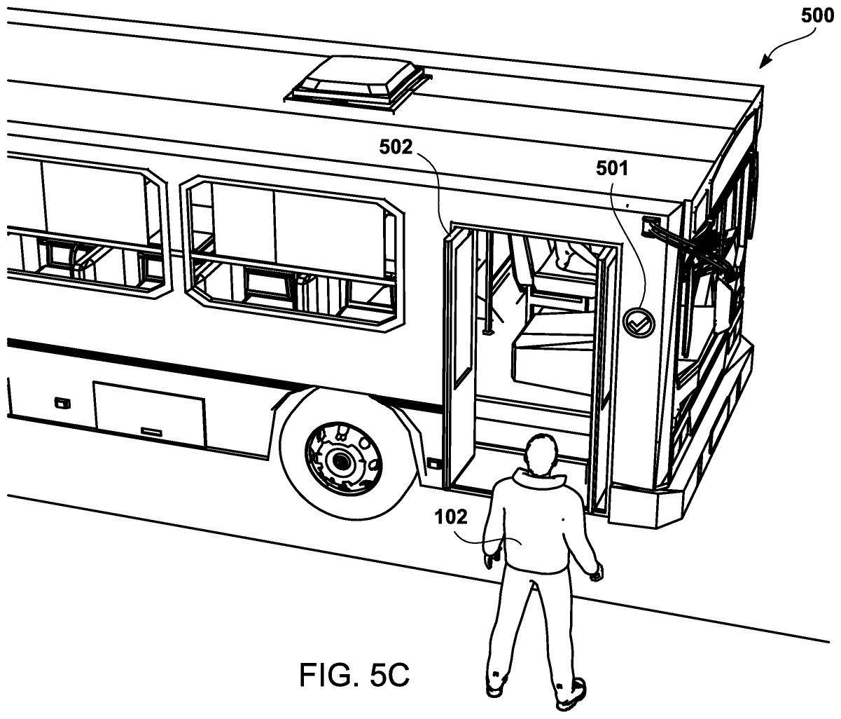

Irrespective of the particular dual-biometric configuration selected for implementation, a dual-biometric configuration is used to identify a user and determine health symptom compliance prior to granting access to the user 102 to products and/or services in a variety of contexts. FIGS. 5A-5C illustrate a particular example in the transportation context. In particular, FIG. 5A illustrates a bus 500, which initially has restricted access, as indicated by an access indicium 501 (e.g., image, sign, text, etc. indicative of access restriction). As the user 102 approaches the bus 500, the door 502 is closed, and remains closed until the user 102 provides dual biometric validation of his identity and health. Accordingly, as an example, the user 102 may provide his fingerprint on the mobile computing device 101. Upon validation, local or remote, of the identity of the user 102, the mobile computing device 101 may transmit the identity of the user 102 to a computing device positioned in the bus 500. Alternatively, the mobile computing device 101 performs the biometric validation without transmission of the identity of the user 102 to the computing device on the bus 500.

Furthermore, FIG. 5B illustrates the user holding the mobile computing device 101 to his ear to perform a preliminary health check. For example, the mobile computing device 101 may have an IR emitter, which emits IR light toward the eardrum of the user 102 while the user 102 is holding the phone close to his ear (similar to a hand position for speaking on the telephone). The mobile computing device 101 may also have an IR detector that detects, and measures the heat, of the IR light that reverberates from the eardrum of the user 102. Accordingly, the mobile computing device 101 is able to perform health measurements for a particular user prior to that user being granted access on the bus 500. Although the temperature of the user 102 is illustrated as being taken via an ear measurement, other forms of measurement (e.g., forehead) via other types of devices (e.g., smart accessory) may be performed instead.

In one embodiment, the particular types of health measurements performed by the mobile computing device 101 may be adjusted on-the-fly given a particular health occurrence within a given community. For example, within a given community at a particular time of year, health officials may declare an influenza pandemic. Accordingly, the application server 104, with which the mobile computing device 101 is in remote communication, or an access computing device, which may positioned on the bus 500 for proximity-based wireless communication with the mobile computing device 101, may be configured to request specific health measurements, corresponding to symptoms for that particular strain of influenza, be performed by the mobile computing device 101. Yet, at another time of the year, the mobile computing device 101 may be configured to perform health measurements, via one or more sensors 404, commensurate with a less innocuous, but inconvenient, health concern such as the common cold. Therefore, the dual-biometric configuration of the mobile computing device 101 may perform dynamic adjustments to measure health symptoms, given a particular health concern.

Only upon compliance with the given health criteria at a given time does the door 502 of the bus 500 automatically open to grant access to the user 102, as illustrated in FIG. 5C. Furthermore, the access indicium 501 may change (e.g., imagery, color, text, etc.) to indicate that access onboard the bus 500 is permitted by the user 102. By measuring symptoms via the mobile computing device 101, communities may minimize the spread of viruses. For example, had the mobile computing device 101 measured a temperature for the user 102 indicative of a symptom of influenza, the user 102 would have been denied access to boarding the bus 500, thereby preventing the exponential spread of the influenza virus that could have easily occurred from just that one person boarding the bus 500.

With any of the dual-biometric configurations provided for herein, various orders of operations may or may not be applicable. In one embodiment, the dual-biometric configuration encompasses a biometric identity validation prior to performing a health check. In another embodiment, the health check may be performed prior to the biometric identity validation. In yet another embodiment, they may be performed simultaneously. In another embodiment, neither the biometric identification data nor the health check data is sent to the product/service provider access device 114, as illustrated in FIGS. 1A-1D, until both have been validated at the mobile computing device 101.

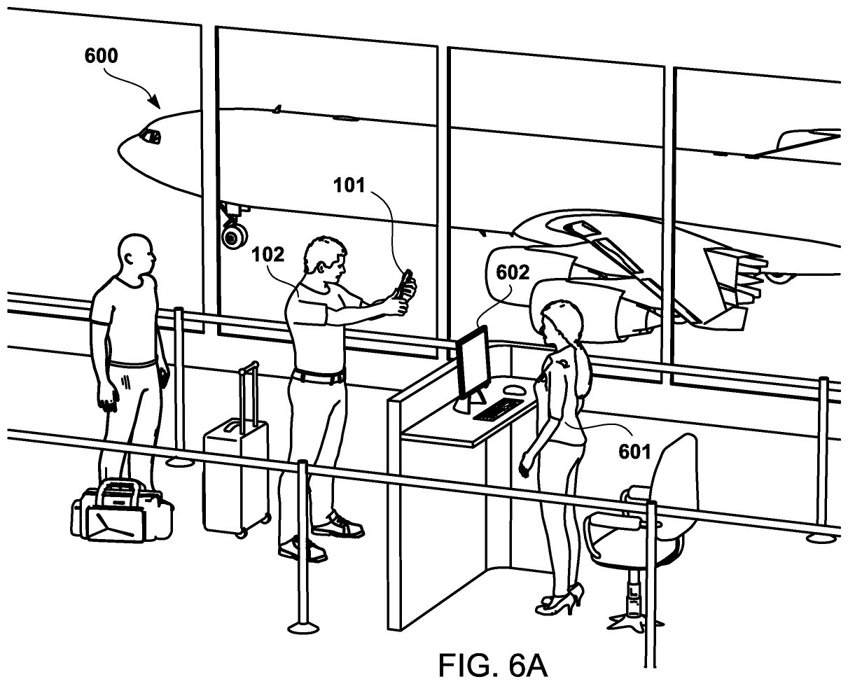

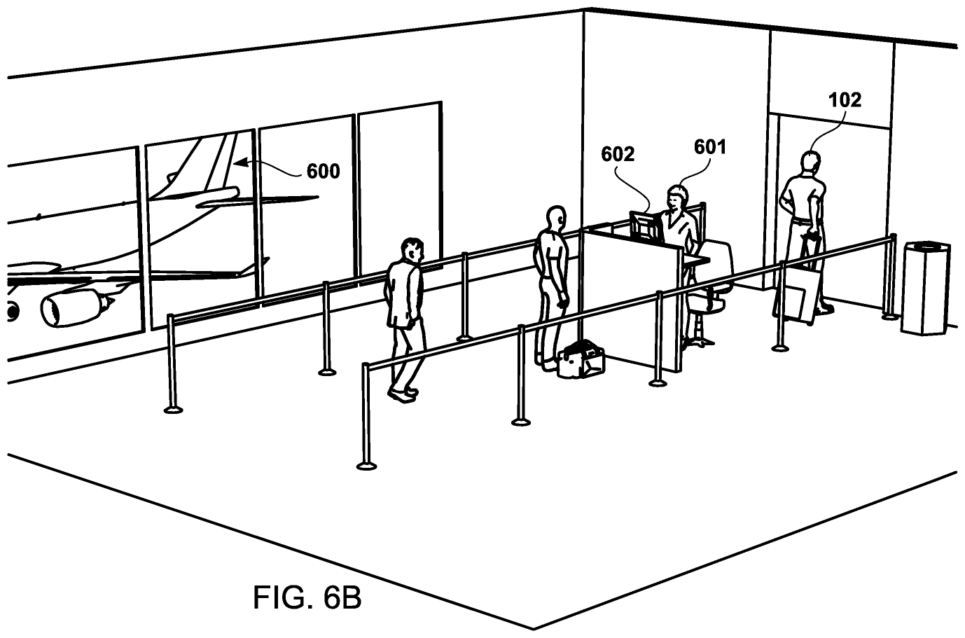

As another example of a dual-biometric configuration within the transportation context, the user 102 may utilize the mobile computing device 101 to board an airplane 600, as illustrated in FIGS. 6A and 6B. In particular, the user 102 may have an e-ticket stored on his mobile computing device 101. Prior to boarding the airplane 600, FIG. 6A illustrates the airline representative 601 asking to view the e-ticket, as well as to perform biometric validation of the identity of the user 102. In one embodiment, the computing device 602 of the airline representative 601 receives biometric identification data along with e-ticket information, allowing the airline representative 601 to automatically validate the identity of the user 102 upon the user providing a biometric input at the mobile computing device 101. Furthermore, the mobile computing device 101 of the user 102 may send health measurements to ensure compliance with health criteria, which may have been determined by the airline or a regulatory entity. Upon compliance with both the identification and health validation, the user 102 is cleared to board the airplane 600, as illustrated in FIG. 6B.

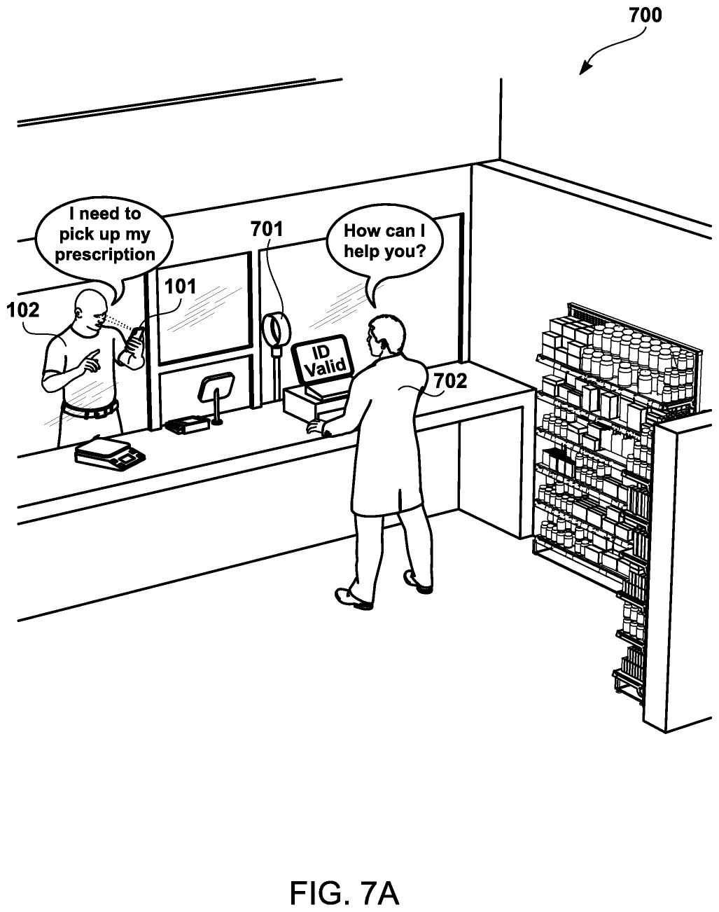

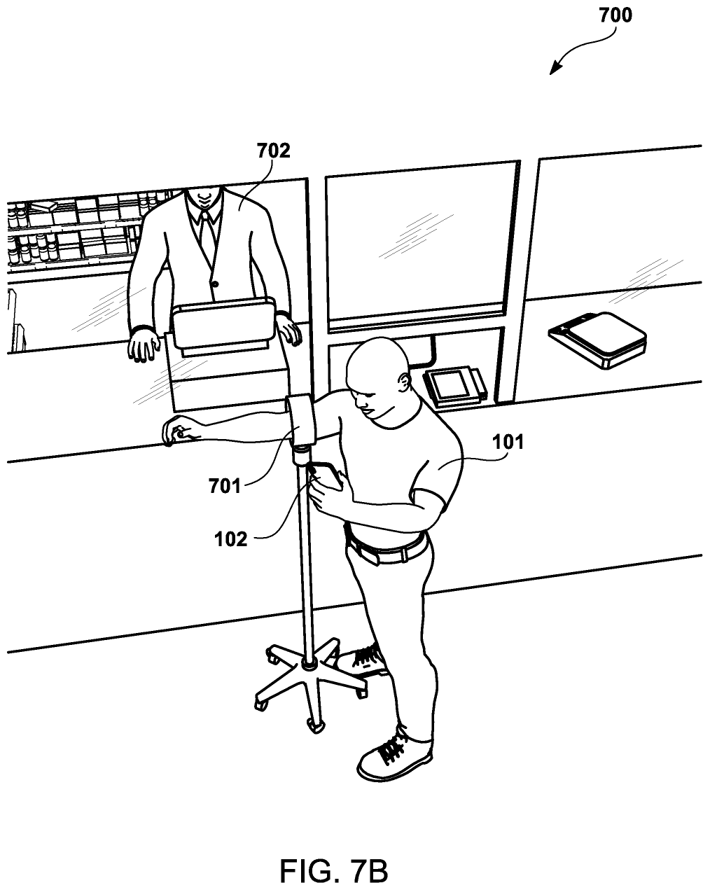

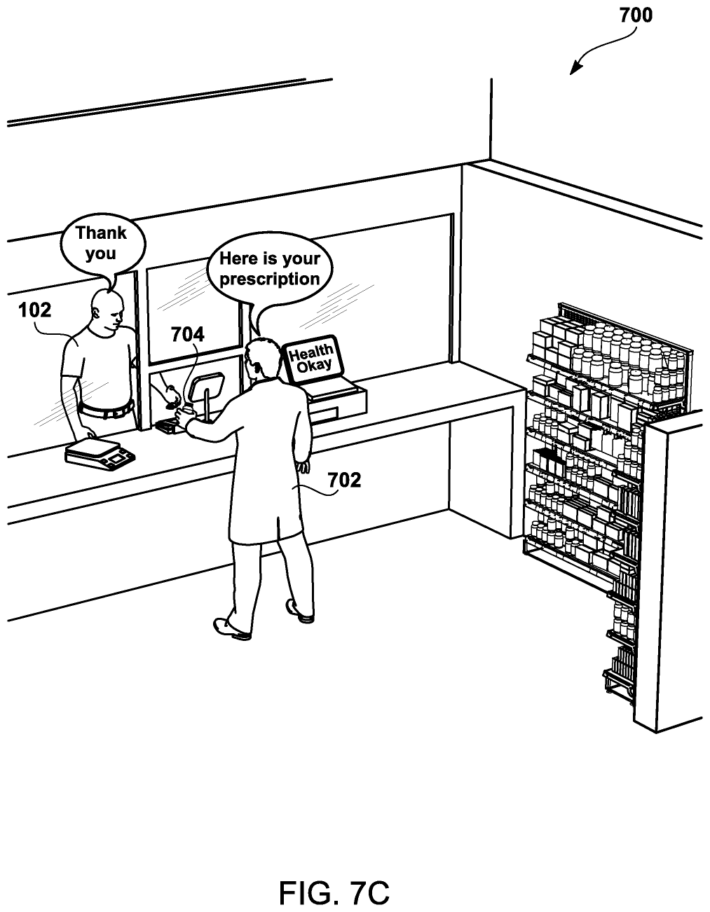

In addition to preventing the spread of viruses, the dual-biometric configurations provided for herein may be utilized to manage distribution of medications, especially those that are highly addictive. FIGS. 7A-7C illustrate a pharmacy 700 in which the user 102 requests pickup of his medication. In particular, FIG. 7A illustrates the user 102 validating his identity, via a biometric input (e.g., iris scan) at the mobile computing device 101. Furthermore, FIG. 7B illustrates the user 102 using a statically-positioned blood pressure monitoring device 701 to measure his blood pressure. (Other types of devices and measurements may be used instead.) Given that acceptable health measurements may be vary by an individual's particular biological traits (e.g., age, height, weight, etc.), the mobile computing device 101 may wirelessly receive the health measurement data to perform a comparison with the received data. For example, the mobile computing device 101 may store electronic medical records from the physician of the user 102. The electronic medical records may indicate acceptable ranges of health measurements for the user 102. Finally, as illustrated in FIG. 7C, the pharmacist 702 may provide the medication 704 to the user 102 upon the biometric validation criteria being met.

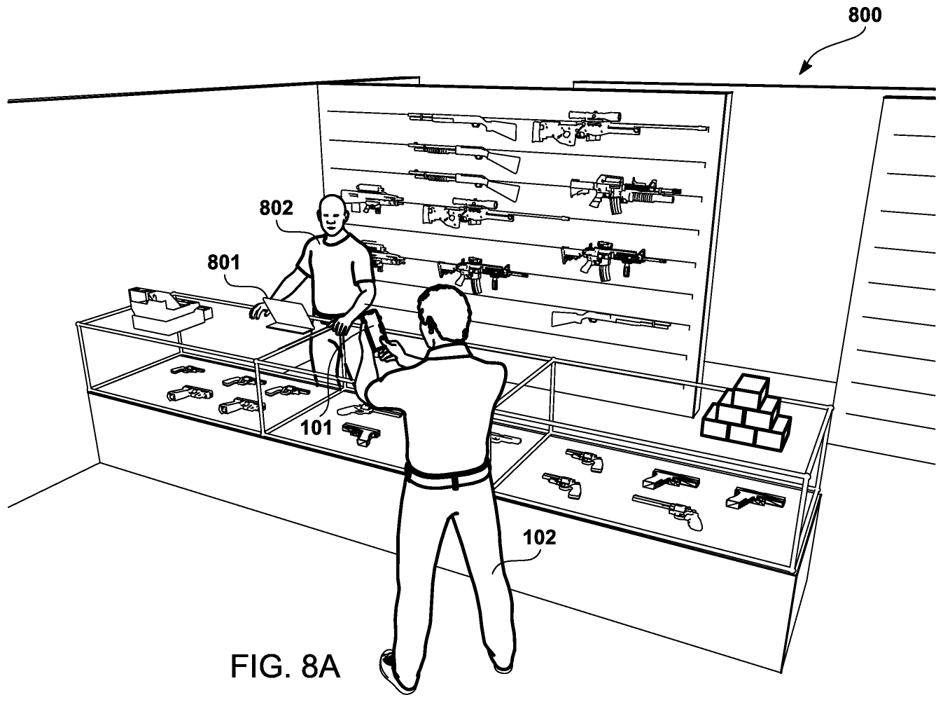

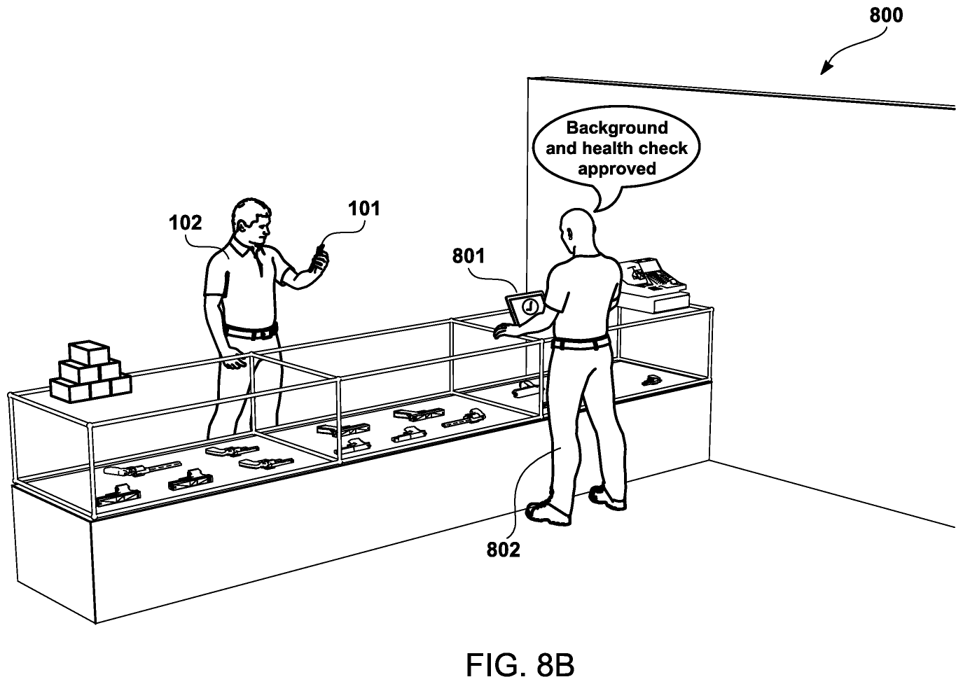

Moreover, the dual-biometric configurations provided for herein may be utilized in addition to background checks to manage the sale and distribution of controlled products, such as firearms. FIGS. 8A and 8B illustrate a firearm distribution environment 800 (e.g., gun shop, gun show, etc.) in which a firearm representative 802 may offer various firearms for sale. In particular, FIG. 8A illustrates the user 102 entering the firearm distribution environment 800 to purchase a firearm. Prior to purchasing the firearm, the user 102 may have to provide certain biometric identification information, such as a thumbprint. Accordingly, the user 102 may place his or her thumb on the touch screen of the mobile computing device 101 to provide a user input with the thumbprint of the user 102. The mobile computing device 101 may wirelessly communicate with a POS device 802 to provide the thumbprint of the user 102. As a result, the POS device 802 may perform a background check on the user 102. Furthermore, the mobile computing device 101 may send health biometric data as a result of that one thumb user input (e.g., pulse rate), and/or additional user inputs corresponding to other health biometric data, so that the POS device 801 may perform an additional assessment on the user 102. In other words, even if the user 102 successfully complies with the background check, the firearm distribution environment 800 may consider the health of the user 102 prior to completing the sale of the firearm to prevent potential firearm casualties. For instance, the physical symptoms of the user 102 may reflect the current mental state of the user 102, which may or may not be commensurate with a user that should be allowed to purchase a firearm. As illustrated in FIG. 8B, the POS device 801 may indicate that the identification and health validations were successful, thereby allowing the firearm representative 802 to proceed with the sale of the firearm to the user 102. In one embodiment, the firearm distribution environment 800 may have one or more electronic locks that prohibit access to the firearm, even by the firearm representative 802, until the biometric validations are successfully indicated by the POS device 801. Accordingly, the dual-biometric configurations provided for herein may be utilized for the process not only of selling a firearm, but also for showing a firearm to a customer that is potentially interested in a firearm purchase.

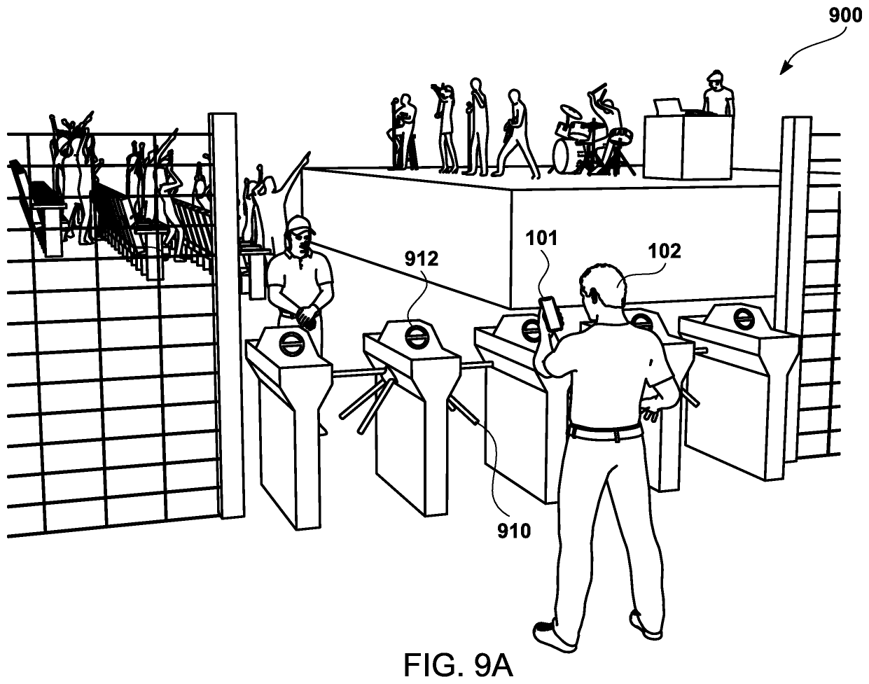

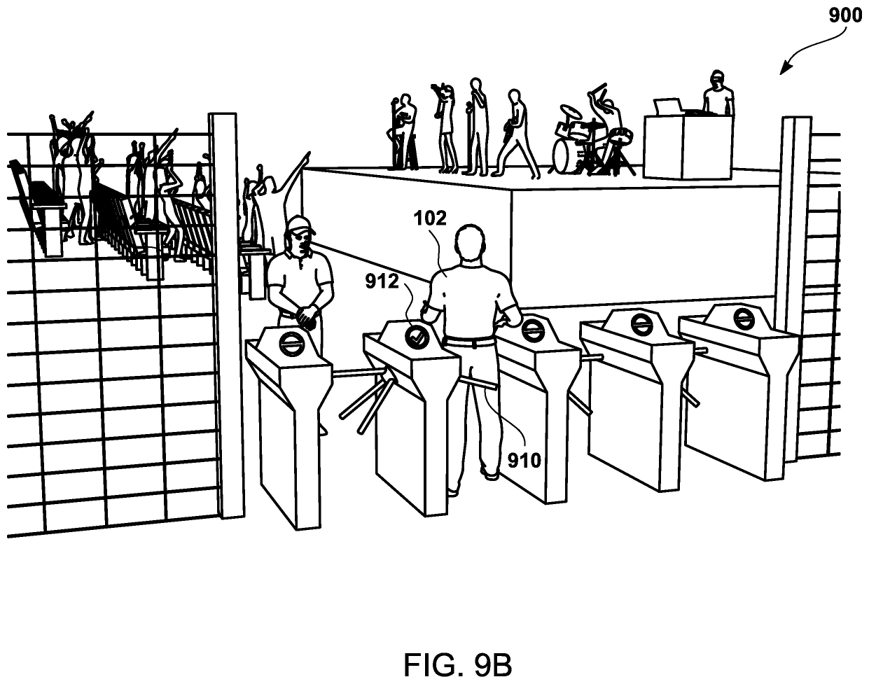

The dual-biometric configurations are not limited to one-on-one interactions with other humans, but may also provide health risk minimization in crowded areas, such as the following: conventions, conferences, classrooms, concerts, sporting events, workplace meetings, hotels, rental properties, condominiums, etc. As an example, FIGS. 9A and 9B illustrate the dual-biometric configuration being implemented in a concert environment 900. In particular, FIG. 9A illustrates the user 102 approaching a turnstile to obtain access to a concert. In one embodiment, the user 102 may wirelessly provide identification information (e.g., e-ticket) to an access system built into the turnstile 910 (e.g., via NFC, barcode, QR code, etc.). Prior to providing such identification information, the user 102 may have to perform biometric validation (e.g., thumbprint, fingerprint, etc.). Furthermore, the user 102 may have to provide health data (via the user mobile computing device 101, an accessory device, or a stationary device positioned at the concert) to obtain access via the turnstile 910. In one embodiment, an access indicium (e.g., image, sign, etc.) is positioned on, or in proximity to, the turnstile 910 to indicate whether the user 102 is allowed access through the turnstile 910 to the concert. As illustrated in FIG. 9B, the user 102 has successfully completed the biometric identification and biometric health check, and, therefore, is automatically permitted access through the turnstile 910, as indicated by a change in the access indicium 912.

The automated restricted access system illustrated in FIGS. 9A and 9B not only minimizes the risk of viruses to other human beings in the concert, but also eliminates the possibility of staff potentially getting the virus. In other words, the automated restricted access system may be implemented without human beings monitoring tickets and health of individuals at the concert.

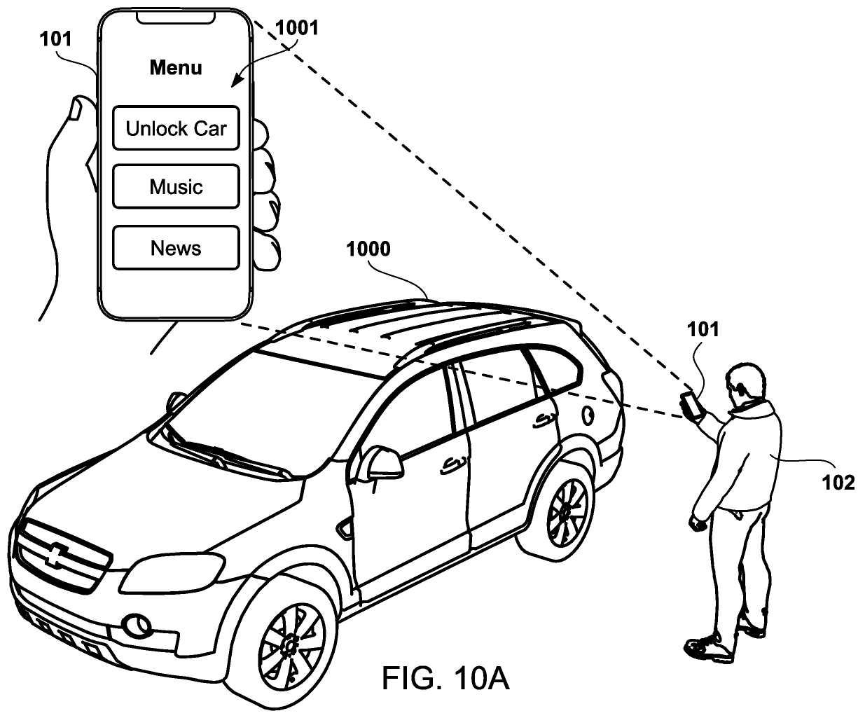

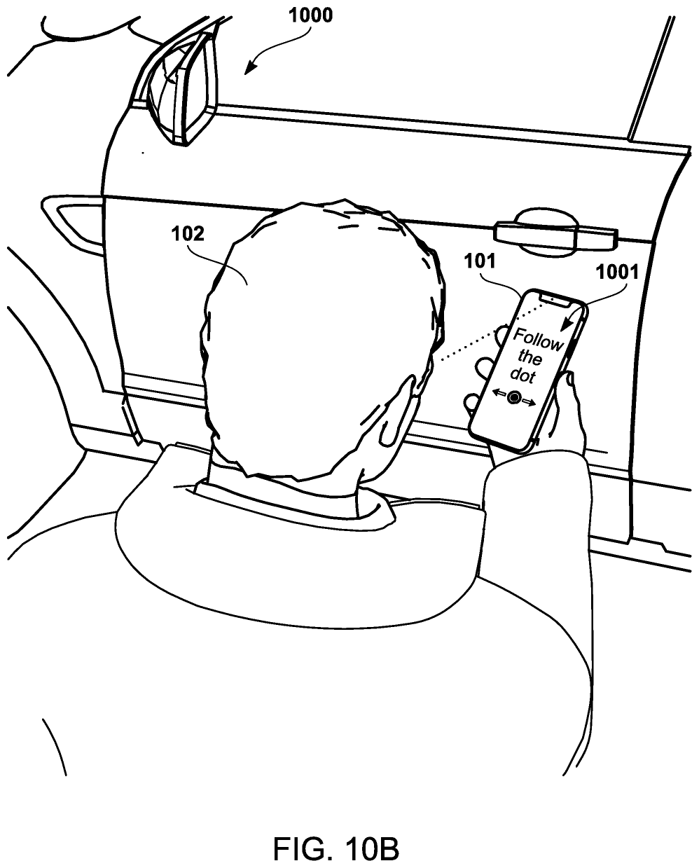

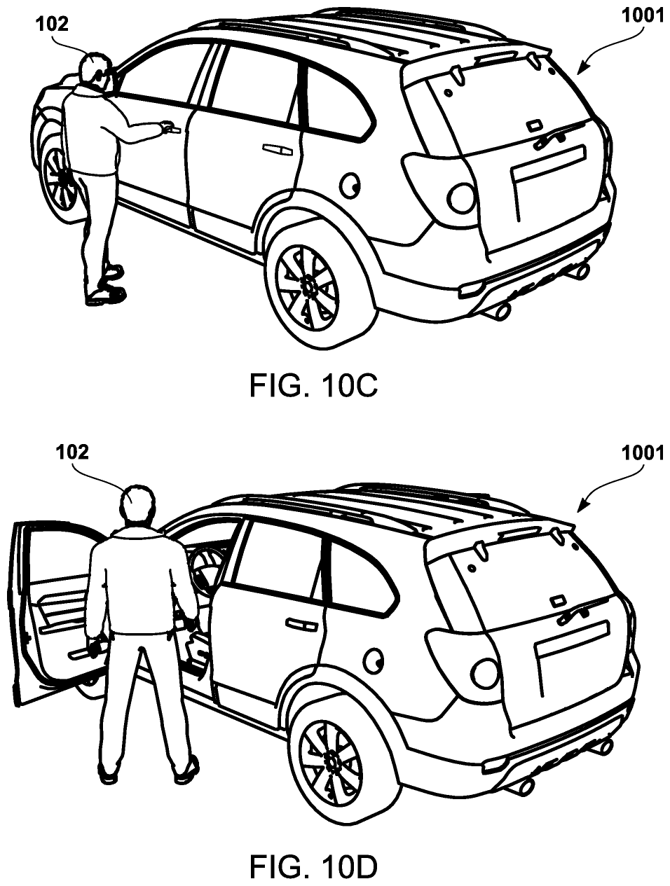

Furthermore, the dual-biometric configurations provided for herein may be utilized to restrict access to an automobile 1000, as illustrated in FIGS. 10A-10C. Whether the automobile belongs to the user 102, or is temporarily leased to the user 102, the dual-biometric configuration prevents the user 102 from accessing the automobile 1000 unless he or she meets one or more health criteria. In particular, FIG. 10A illustrates the user 102 utilizing a GUI 1001 on the mobile computing device 101 to access a menu to unlock the automobile 1000. Initially, the user 102 may provide a biometric input that identifies the user 102 as the owner or lessee of the automobile 1000. In an alternative embodiment, the user 102 may have a key that allows the user 102 to access the automobile 1000 without a biometric identification, but the user 102 would still have to comply with a biometric health check. As an example of the biometric health check, as illustrated in FIG. 10B, the mobile computing device 101 may be configured to render a visual game, which tests the reflexes of the user 102. For example, the user 102 may have to move his or her eyes back and forth in various directions. An image capture device 406, illustrated in FIG. 4, of the mobile computing device 101 may then capture the eye movements of the user 102 to detect alertness of the user 102. By detecting such alertness, the mobile computing device 101 may prevent the user 102 from operating the automobile 1000 if the user 102 is intoxicated, sick, or not physically prepared in some other manner for operating the automobile 1000 at that given moment. FIG. 10C illustrates the user 102 attempting to open a door of the automobile 1000, after successful biometric identification and health check validation. Finally, FIG. 10D illustrates the user 102 successfully opening the door of the automobile 1000.

In another embodiment, the user 102 is permitted access to the automobile 1000 (e.g., to provide protection from inclement weather), but is not able to operate it (e.g., the ignition is prevented from being started) until successful biometric identification and health check compliance have been completed.

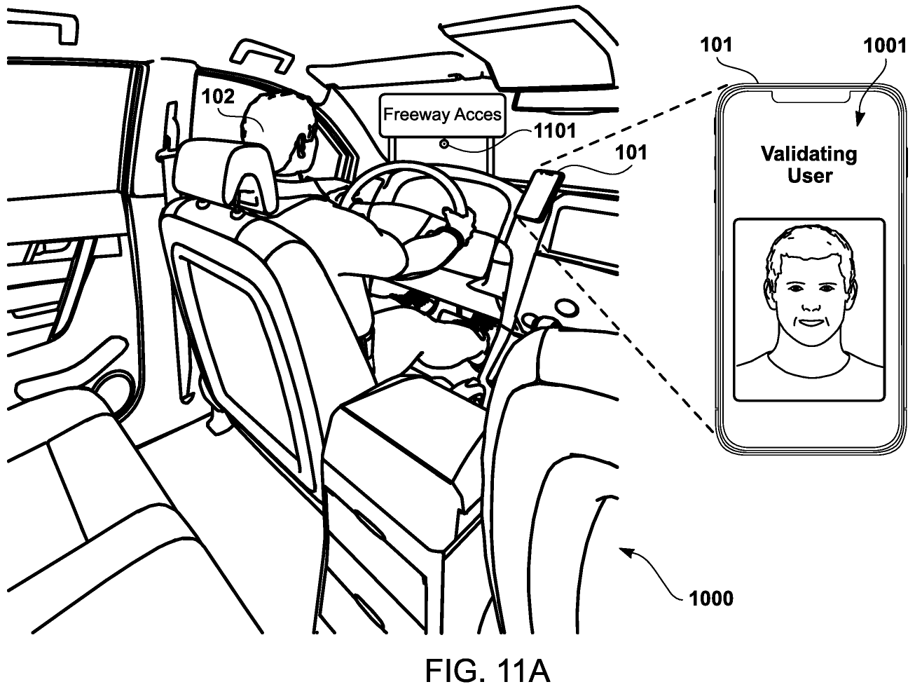

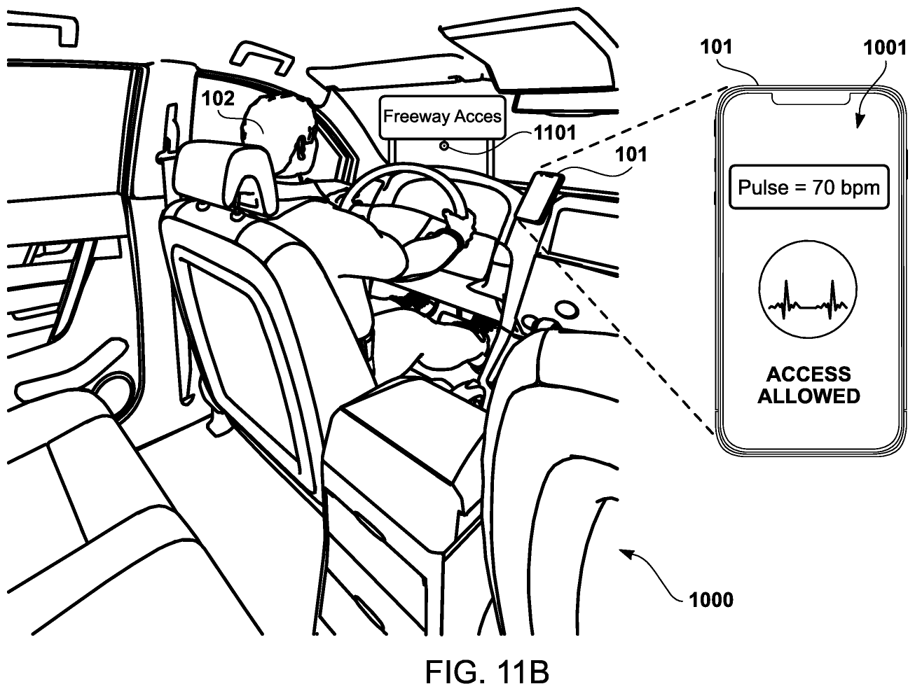

Furthermore, even after accessing an automobile, the user 102 may potentially develop symptoms that are not conducive to normal driving habits. Accordingly, as illustrated in FIGS. 11A and 11B, the dual-biometric configurations provided for herein may be utilized to restrict access by the user 102 to various transportation infrastructure, such as roads, bridges, bridges, and tunnels. For instance, FIG. 11A illustrates the user 102 in the automobile 1000 driving toward a freeway access point. Furthermore, the mobile computing device 101 may be configured (e.g., via a transponder) to communicate with an access point device 1101 via wireless transmission to indicate the identity of the user 102 and/or automobile 1000 for the purpose of obtaining freeway access. To ensure that the user 102 is actually the user 102, not a person that unscrupulously stole the mobile computing device 101, the mobile computing device 101 may perform a biometric identification validation of the user 102. For example, to avoid distracting the user 102 while driving, the mobile computing device 101 may be configured to automatically capture imagery of the user 102 to perform facial recognition without a manual input from the user 102, upon being within a predetermined proximity to the access point device 1101. For example, the access point device 1101 may send a signal to the mobile computing device 101 requesting the mobile computing device 101 perform the facial recognition of the user 102. Furthermore, as illustrated in FIG. 11B, a biometric health check of the user 102 may be performed to ensure that the user 102 is in a condition to drive the automobile 1000. For example, the user 102 may wear the smart bracelet 201, illustrated in FIG. 2A, to detect the health parameters (e.g., pulse rate) of the user 102. Again, the smart bracelet 201 may be automatically configured to detect such data without a manual input from the user 102 to avoid distracting the user 102 while driving the automobile 1000. Additionally, the smart bracelet 201 may wirelessly communicate with the mobile computing device 101, which may then transmit the health check data to the access point device 1101 to obtain access to the freeway for the user 102. Alternatively, the mobile computing device 101 may perform the health check (e.g., image capture of pupil dilation). As yet another alternative, the access point device may be in operable communication with a stationary health measurement device, positioned at an infrastructure checkpoint, that performs the health measurement of the user 102, and automatically communicates the health check data to the access point device.

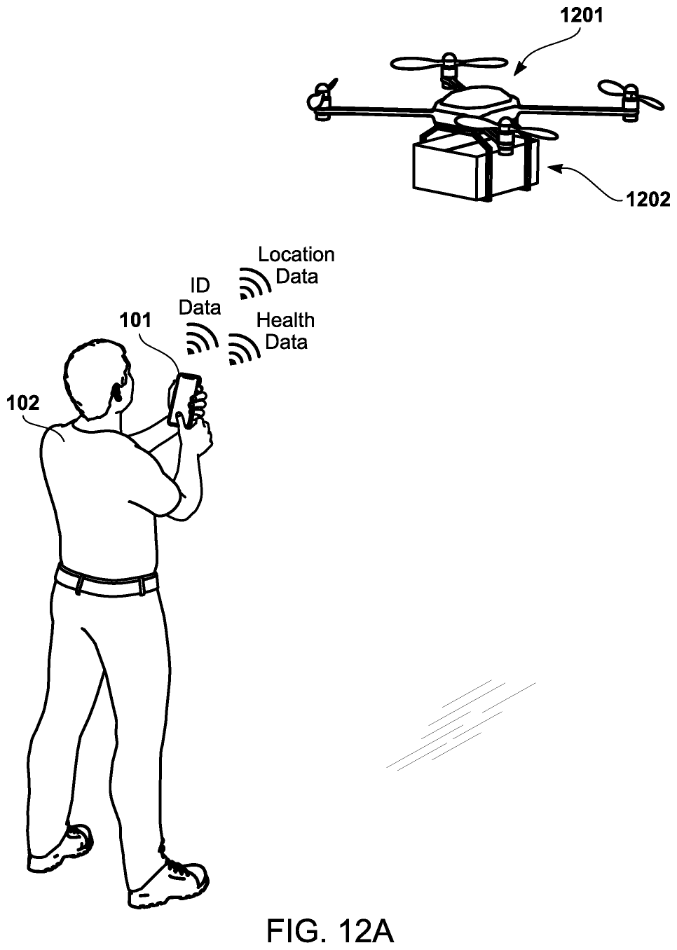

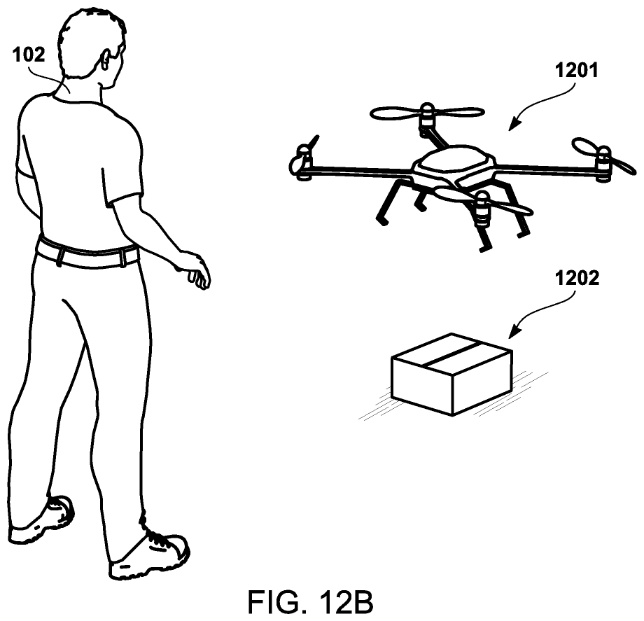

To provide further convenience to the user 102, while minimizing human interaction, the dual-biometric configurations provided for herein may be utilized to interact with a UAV 1201 (e.g., drone) that delivers a package 1202 to a geographical location of the user 102, as illustrated in FIGS. 12A and 12B. In particular, FIG. 12A illustrates the user 102 utilizing the mobile computing device 101 to emit location data, biometric identification data, and biometric health data to the UAV 1201. Based on the location determined by an integrated location detection device 405, the mobile computing device 101 is able to provide the location to the UAV 1201 to which the UAV 1201 should bring the package 1202. Depending on the particular contents of the package 1202 (e.g., prescription medication), the UAV 1201 may necessitate biometric identification and a biometric health check of the user 102. In one embodiment, upon being within proximity to the mobile computing device 101, the UAV 1201 may transmit a message to the mobile computing device that prompts the mobile computing device 101 to request one or more biometric inputs from the user 102 via a software application. For example, the user 102 may provide a thumbprint that identifies the user 102 and performs a health check based on the pulse of the user 102. Upon successful biometric identification and biometric health check validation, the UAV 1201 may deliver the package 1202 to the user 102, as illustrated in FIG. 12B.

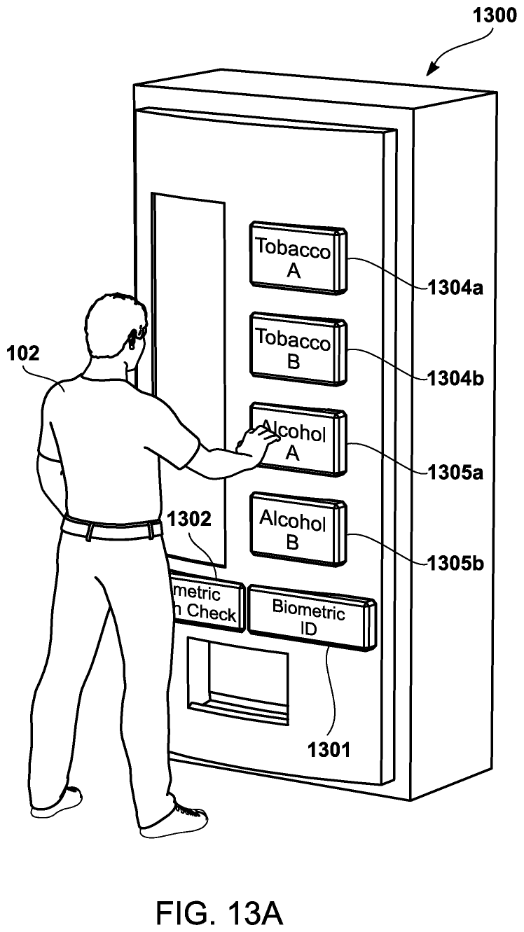

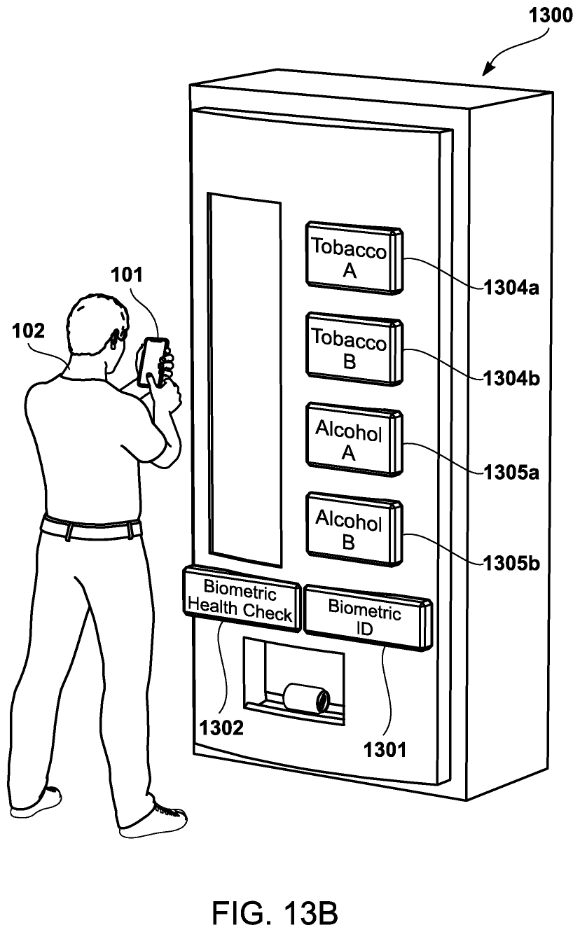

As another example, FIGS. 13A and 13B illustrate a vending machine 1300 that may be utilized to automatically dispense items, such as controlled items, based upon biometric identification and biometric health check validation. For example, FIG. 13A illustrates the user 102 approaching the vending machine 1300 to provide biometric identification. The user 102 may select a controlled product (e.g., a tobacco product 1304a or 1304b, an alcohol product 1305a or 1305b, etc.), and then may press a biometric identification indicium 1301 (e.g., button) to initiate biometric identification. For example, the user 102 may have to meet a particular age requirement, which may be confirmed via a biometric input, such as a thumbprint, being compared by the vending machine 1300 with a biometric input database that is correlated to age. Alternatively, the user 102 may provide the biometric input to allow the mobile computing device 101 to provide an image of a valid form of identification (e.g., driver's license) to the vending machine 1300. Upon successful biometric identification, the user 102 may select a biometric health check indicium (e.g., button) 1302 on the vending machine 1300. Accordingly, the user 102 may then utilize the mobile computing device to determine one or more health parameters. Alternatively, the vending machine 1300 may have one or more health measurement devices that the user 102 may use to perform the biometric health check. Upon compliance with the one or more health criteria, the user 102 may obtain his or her product from the vending machine 1300, as illustrated in FIG. 13B.

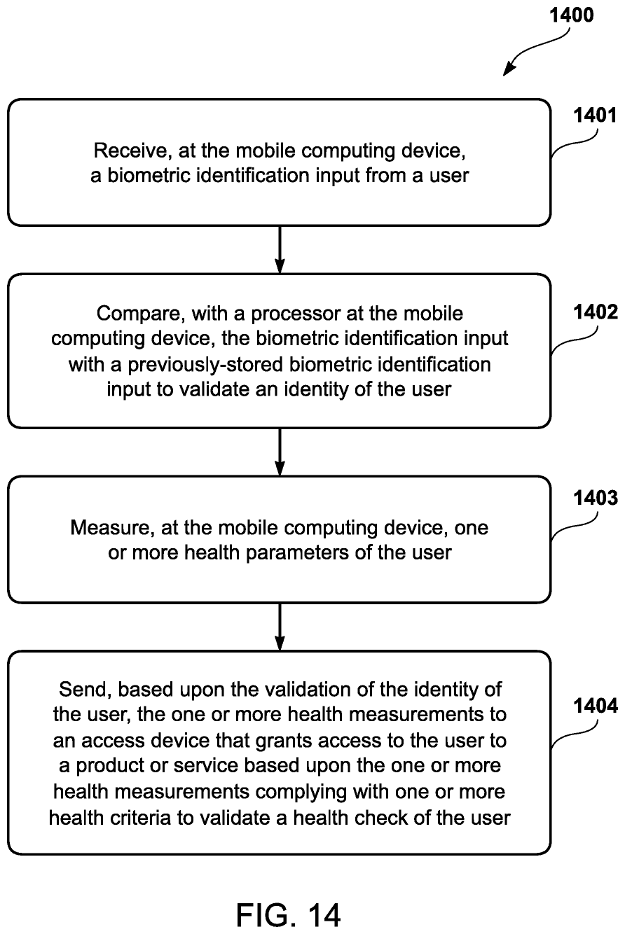

FIG. 14 illustrates a process 1400 that may be utilized to perform a dual-biometric configuration. At a process block 1401, the process 1400 receives, at the mobile computing device 101, a biometric identification input from a user 102. Furthermore, at a process block 1402, the process 1400 compares, with a processor 401 at the mobile computing device 101, the biometric identification input with a previously-stored biometric identification input to validate an identity of the user 102. Additionally, at a process block 1403, the process 1400 measures, at the mobile computing device 101, one or more health parameters of the user. Finally, at a process block 1404, the process 1400 sends, based upon the validation of the identity of the user 102, the one or more health measurements to an access device that grants access to the user 102 to a product or service based upon the one or more health measurements complying with one or more health criteria to validate a health check of the user 102.

In another embodiment, the process 1400 may be implemented to send the biometric identification input to a server, rather than perform the biometric validation via the mobile computing device.

In yet another embodiment, the process 1400 may be implemented such that the health measurements are provided to an entity without biometric identification data. Accordingly, the mobile computing device 101 may perform the biometric identification of the user 102, but the health measurements may be analyzed by a server independently of having biometric identification information of the user 102, thereby protecting the privacy of the user 102.

Furthermore, the dual-biometric configurations provided for herein are not limited to obtaining access to a product or service. Upon performing biometric identification and health check validation, the mobile computing device 101 of the user 102 may provide health check updates to one or more remotely situated devices, associated with person to whom such information is of interest (e.g., family member, friend, physician, work colleague, team member, etc.).

It is understood that the processes, systems, apparatuses, and computer program products described herein may also be applied in other types of processes, systems, apparatuses, and computer program products. Those skilled in the art will appreciate that the various adaptations and modifications of the embodiments of the processes, systems, apparatuses, and computer program products described herein may be configured without departing from the scope and spirit of the present processes and systems. Therefore, it is to be understood that, within the scope of the appended claims, the present processes, systems, apparatuses, and computer program products may be practiced other than as specifically described herein.

I claim:

1. A computer program product comprising a non-transitory computer useable storage device having a computer readable program, wherein the computer readable program when executed on a mobile computing device causes the mobile computing device to:
receive, at the mobile computing device, a biometric identification input from a user;
compare, with a processor at the mobile computing device, the biometric identification input with a previously-stored biometric identification input to validate an identity of the user;
measure, at the mobile computing device, one or more health parameters of the user; and
send, based upon the validation of the identity of the user, the one or more health measurements to an access device that grants access to the user to a product or service based upon the one or more health measurements complying with one or more health criteria to validate a health check of the user.

2. The computer program product of claim 1, wherein the mobile computing device is further caused to perform the measurement of the one or more health parameters in real-time with the reception of the biometric identification input.

3. The computer program product of claim 1, wherein the mobile computing device is further caused to perform the measurement of the one or more health parameters in simultaneity with the reception of the biometric identification input.

4. The computer program product of claim 1, wherein the biometric identification input is selected from the group consisting of: an iris scan, facial recognition, a thumbprint, a fingerprint, and palm scan.

5. The computer program product of claim 1, wherein the one or more health parameters are selected from the group consisting of: temperature, pulse rate, blood pressure, pupil dilation, and sweat quantity.

6. The computer program product of claim 1, wherein the mobile computing device is further caused to perform the measurement of the one or more health parameters based on the reception of the biometric identification input.

7. The computer program product of claim 1, wherein the mobile computing device is further caused to detect, with a proximity-based module integrated within the mobile computing device, proximity to a proximity-based reader positioned within the access device, the proximity-based reader being positioned externally to the mobile computing device.

8. The computer program product of claim 7, wherein the proximity-based transmission module is an NFC physical circuit.

9. The computer program product of claim 7, wherein the proximity-based transmission module is an NFC logical circuit.

10. A process comprising:
receiving, at a mobile computing device, a biometric identification input from a user;
measuring, at the mobile computing device, one or more health parameters of the user;
sending, with a processor at the mobile computing device, the biometric identification input to an access device to obtain identification validation of the user; and
sending, with the processor at the mobile computing device, the one or more health measurements to the access device, the access device granting access to the user to a product or service based upon the identification validation of the user and the one or more health measurements complying with one or more health criteria to validate a health check of the user.

11. The process of claim 10, further comprising performing the measurement of the one or more health parameters in real-time with the reception of the biometric identification input.

12. The process of claim 10, further comprising performing the measurement of the one or more health parameters in simultaneity with the reception of the biometric identification input.

13. The process of claim 10, wherein the biometric identification input is selected from the group consisting of: an iris scan, facial recognition, a thumbprint, a fingerprint, and palm scan.

14. The process of claim 10, wherein the one or more health parameters are selected from the group consisting of: temperature, pulse rate, blood pressure, pupil dilation, and sweat quantity.

15. The process of claim 10, further comprising performing the measurement of the one or more health parameters based on the reception of the biometric identification input.

16. The process of claim 10, further comprising detecting, with a proximity-based module integrated within the mobile computing device, proximity to a proximity-based reader positioned within the access device, the proximity-based reader being positioned externally to the mobile computing device.

17. The process of claim 16, wherein the proximity-based transmission module is an NFC physical circuit.

18. The process of claim 16, wherein the proximity-based transmission module is an NFC logical circuit.

19. An access-based system comprising:
a memory device that stores one or more health criteria associated with a user;
a receiver that receives, from a mobile computing device of a user, one or more health measurements subsequent to a biometric identification validation of the user; and
a processor that compares the one or more health measurements with the one or more health criteria, the processor providing access to the user to a product or service based upon the one or more health measurements complying with one or more health criteria to validate a health check of the user.

20. The access-based system of claim 19, wherein the access-based system performs the biometric identification validation of the user.
